(19) United States
(12) Patent Application Publication
(10) Pub. No.: US 2003/0092053 A1
(43) Pub. Date: May 15, 2003

(54) Storage medium, method for designing genotyping-microarray and computer system containing the same

(76) Inventor: Tae-joon Kwon, Seoul (KR)

Correspondence Address: Michael A. Cantor, 55 Griffin South Road, Bloomfield, CT, 06002, US

(21) Appl. No.: 10/273,789

(22) Filed: Oct. 18, 2002

(30) Foreign Application Priority Data
Nov. 15, 2001 (KR) 2001-71102

(51) Int. Cl. C12Q 1/68; G06F 19/00; G01N 33/48; G01N 33/50

(52) U.S. Cl. 435/6; 702/20

(57) ABSTRACT

Provided are a computer-readable storage medium, a method for designing a genotyping-microarray using the same, and a computer system for designing a genotyping-microarray containing the same. The computer-readable storage medium has stored thereon: a first directory comprising an information on DNA, RNA, protein, and/or genome of a target gene; a second directory comprising an information on a specific region in the target gene; and a third directory containing an information on a probe for identifying the specific region; wherein the first, second, and third directories are organized in a hierarchical structure in which the second directory is at a level lower than that of the first directory and the third directory is at a level lower than that of the second directory.

This application is based upon and claims priority from Korean Patent Application No. 01-71102 filed Nov. 15, 2001, the contents of which are incorporated herein by reference.

### BACKGROUND OF THE INVENTION

1. Field of the Invention

The present invention relates to a computer-readable storage medium, a method for designing a genotyping-microarray using the same, and a computer system for designing a genotyping-microarray containing the same.

2. Description of the Related Art

One of the characteristics in microarray technique is that large quantities of information are concurrently managed. It is very important to manage or analyze the information effectively.

U.S. Pat. No. 6,229,911 discloses a system and method for organizing information relating to polymer probe array chips including oligonucleotide array chips. U.S. Pat. No. 6,188,783 discloses a computer-readable storage medium in systems and method for organizing information relating to a design of polymer probe array chips including oligonucleotide array chips. The storage medium comprises a relational database having a complex inner structure, which contains a probe table including a plurality of probe records and a sequence item table including a plurality of sequence item records. In the relational database, there is a many-to-many relationship between the probe records and the sequence item records.

Conventional systems and method for organizing information relating to a design of polymer probe are suitable for a design of a microarray probe for gene expression profile analysis. Also, systems for designing a microarray of commercially available software/systems relating to bioinformatics are mainly focused on gene expression profile analysis. In these systems, a relational database management system (RDBMS) is used to manage or analyze large quantities of information relating to complicated genetic networks.

However, in case of a genotyping-microarray that identifies a genetic variation or determines existence of a specific gene, RDBMS-based systems are too complicated to be applied. That is, experimental data directly affecting design of a genotyping-microarray and/or analysis on results thereof are not so various as to be managed in the form of database. Further, if a target gene is changed, these experimental data need not be used again.

Moreover, in designing a genotyping-microarray for identification of a genetic variation, additional application library (for example, object-relational database system) is required to organize in form of a relational database large quantities of the related information on a target gene, a specific region of the target gene, a genetic variation, and a probe for identifying the specific region.

Therefore, what is needed is a system and method suitable for effectively storing and organizing large quantities of information used in conjunction with a genotyping-microarray design.

### SUMMARY OF THE INVENTION

The present invention provides a computer-readable storage medium in which large quantities of information for genotyping-microarray probe design are stored to be promptly and easily accessible.

Further, the present invention provides a method for designing a genotyping-microarray using the same and a computer system for designing a genotyping-microarray containing the same.

In one aspect of the present invention, there is provided a computer-readable storage medium having stored thereon: a first directory comprising an information on DNA, RNA, protein, and/or genome of a target gene; a second directory comprising an information on a specific region in the target gene; and a third directory comprising an information on a probe for identifying the specific region, wherein the first, second, and third directories are organized in a hierarchical structure in which the second directory is at a level lower than that of the first directory and the third directory is at a level lower than that of the second directory.

In another aspect of the present invention, there is also provided a method for designing a genotyping-microarray comprising: operating the computer-readable storage medium to obtain an information on a plurality of probes; selecting a probe having a desired characteristic, based on the obtained probe information; and forming an microarray comprising the selected probe.

In still another aspect of the present invention, it is provided a computer system for designing a genotyping-microarray comprising a processor and a computer-readable storage medium accessible by the processor.

### BRIEF DESCRIPTION OF THE DRAWINGS

The above objects and advantages of the present invention will become more apparent by describing in detail preferred embodiments thereof with reference to the attached drawings in which:

FIG. 1 is a schematic view of a computer system suitable for executing the present invention;

FIG. 2 illustrates a directory structure comprising a basic information on a disease;

FIG. 3 illustrates a directory structure comprising large quantities of biological information relating to a gene item (an information on DNA, RNA, protein, and/or genome); and

FIG. 4 illustrates a directory structure comprising an information on a target gene, a genetic variation, and a probe.

### DETAILED DESCRIPTION OF THE INVENTION

A computer-readable storage medium of the present invention includes information on a target gene, a specific region in the target gene, a probe for identifying the specific region in directories organized in a hierarchical structure depending on a type of information. That is, the computer-readable storage medium has stored thereon: a first directory comprising an information on DNA, RNA, protein, and/or genome of a target gene; a second directory comprising an information on a specific region in the target gene; and a third directory comprising an information on a probe for identifying the specific region. The first, second, and third directories are organized in a hierarchical structure in which the second directory is at a level lower than that of the first directory and the third directory is at a level lower than that of the second directory.

A target gene may be a portion or an entire portion of a gene relating to a disease. Therefore, one or more target genes may be selected for one gene relating to a disease. The storage medium of the present invention may contain information on one or more target genes.

A DNA item may include information obtained by sequencing a genomic DNA. This item generally includes an information on exon, intron, promoter, etc., an information on genetic variations, and/or an information on DNA sequence. Some of the information may be obtained from actual experiments and others may be obtained from public databases. Therefore, this item may also include an information on original databases from which the information is obtained, key values for searching for the information from such databases, and references relating to genetic information.

An RNA item may include a genetic information expressed to RNA, such as information on EST. Further, this item may include an information on genetic variations, base sequences, transcription, related databases, and references.

A protein item is helpful for determining whether or not a specific genetic variation will cause a fatal effect. In this item, may be included an information on an amino acid replacement by a genetic variation and a protein structure as well as an amino acid sequence. Where a target gene encodes an enzyme, an amino acid replacement occurred in an active site of the enzyme may be recognized as a fatal variation. Further, this item may include information on references and other databases relating to an expressed protein.

A genome item includes information based on draft of the human genome project. This item may include an information on an STS and a locus which makes it possible to identify the locus of a gene in whole chromosome. Further, this item may include an information on base sequence, transcription, genetic variations, related databases, and references.

The specific region in the target gene includes a variation region, where a genetic variation, such as substitution, insertion, and deletion, is occurred.

The information on a probe for identifying the specific region may include information on a base sequence of the probe, a hybridization simulation result, and/or a thermodynamic characteristic of the probe. The thermodynamic characteristics preferably include Tm (melting temperature), cross hybridization, self-dimer formation energy, hairpin formation energy, etc.

The directories may be embodied in form of a computer-readable code in a computer-readable storage medium. A computer-readable storage medium includes any kind of recording media to store computer-readable data. Examples of a computer-readable storage medium include, but not limited to, ROM, RAM, CD-ROM, magnetic tape, floppy disk, and photo-data storage device. Further, the directory may be embodied in form of carrier wave, such as transmission by way of an Internet. Also, a computer-readable storage medium may be divided into computer systems that are interconnected by a network, stored in the form of a computer-readable code in divisional methods, and executed.

Where, in carrying out various projects, an integrative management of independent projects is required, directories having a hierarchical structure are more flexible than a relational database. For example, where information on a gene A relating to disease A is organized and then a gene B which is also related to the disease A is newly identified, information on the gene B may be independently collected, stored, and then combined into a level lower than that of the disease A for integrative and systematic management.

In designing a genotyping-microarray using said storage medium of the present invention, an information is collected on DNA, RNA, protein, and/or genome of a target gene relating to a disease and is stored in a first directory.

An information on a genetic variation and a specific region including the genetic variation is collected and stored in a second directory at a level lower than that of the first directory. For example, the second directory includes an information on a variation type, such as substitution, insertion, and deletion, on a variation of a base, and on whether the genetic variation affects a corresponding protein.

At a level lower than that of the second directory, a third directory is organized to include an information on a probe for identifying the specific region. The information on the probe comprises a hybridization simulation result (an information on cross hybridization, melting temperature, etc.), a thermodynamic characteristic of the probe (an information on probe length, hairpin formation energy, self-dimer formation energy, etc.), and/or base sequences of the probe.

By operating a computer-readable storage medium having the information in directories having hierarchical structure, probe information is obtained. From the probe information, a probe having a desired characteristic is selected. In selecting a probe, following factors are considered, such as no cross hybridization, no dimmer-formation, and no hairpin with genes other than the target gene. The selected probe includes a probe for the identification of a wild type gene and a probe for the identification of a mutant type gene in order to identify whether or not the genetic variation of interest exists in the target gene.

By designing a microarray comprising the selected probe in accordance with the present invention, a genotyping-microarray for identifying a genetic variation is fabricated.

In designing a genotyping-microarray from the information on a target gene and a specific region therein, the directories having a hierarchical data structure are easily accessible and manageable compared with relational databases. Where, in carrying out various projects, an integrative management of independent projects is required, directories having a hierarchical structure show more flexible effects than a relational database. As data is stored and organized in directories having a hierarchical structure of gene—genetic variation—probe, it is possible to update data without affecting upper-level data, in case of newly adding or deleting information on any item. In contrast, data update is made in a relational database by updating various tables.

A computer system for designing a genotyping-microarray comprises a processor and a computer-readable storage medium accessible by the processor.

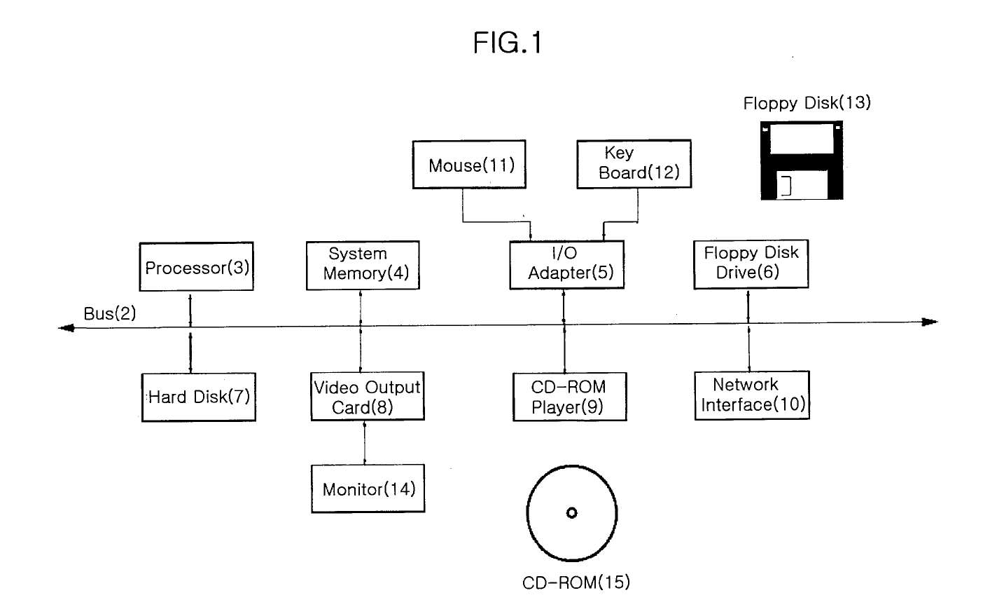

The computer system may be an IBM-compatible personal computer or a workstation, including an appropriate memory and a processor (CPU). FIG. 1 is a schematic view of a computer system suitable for designing a genotyping-microarray. Computer system (1) includes a bus (3) which interconnects a processor (3), a system memory (4) such as RAM, an input/output adapter (5), a mouse (11) and keyboard (12) via an input/output adapter (5), a floppy disk drive (6) operative to receive a floppy disk (13), a hard disk (7), a monitor (14) via a video output card (8), a CD-ROM player (9) operative to receive a CD-ROM (15), and a network interface (10) which may connect to a local area net work (LAN). Many other devices or subsystems may be connected. Further, one or more components shown in FIG. 1 can be omitted to practice the present invention, as discussed below. The devices and subsystems may be interconnected in different ways from those shown in FIG. 1. Explanation for operation of a computer system is omitted. A code to implement a storage medium of the present invention is stored in a computer-readable storage media such as system memory (4), hard disk (7), CD-ROM (15), or floppy disk (13).

Further understanding of the nature and advantages of the present invention herein may be realized by reference to the following Examples. The following Examples are given for the purpose of illustration only, and are not intended to limit the scope of the present invention.

### EXAMPLE

### Diagnosis of the Disease Caused by a Genetic Variation

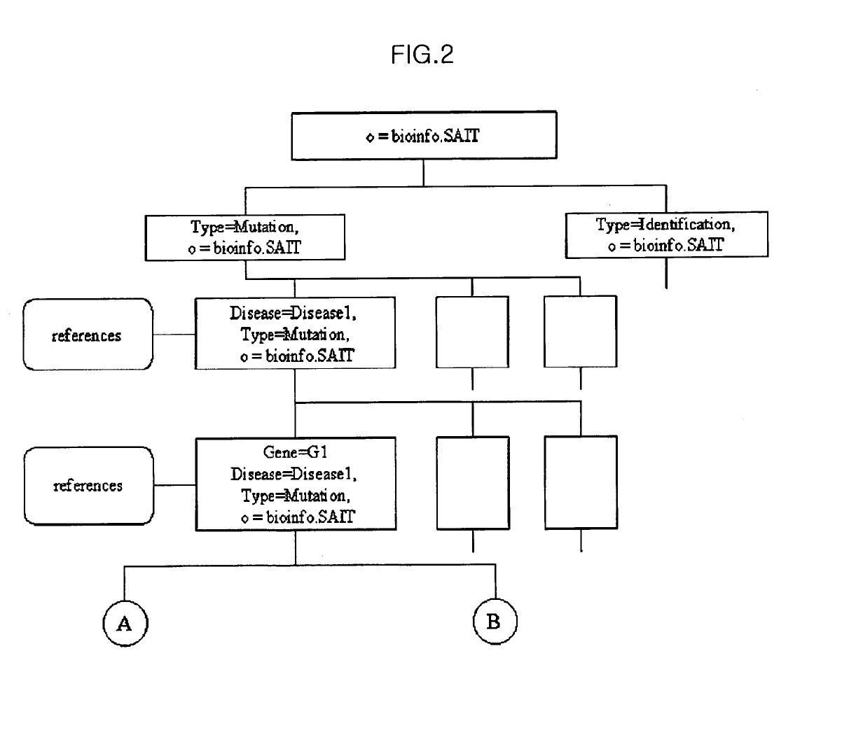

FIG. 2 illustrates a directory structure comprising basic information on diseases. Item o means a highest level of data. “Type” means a type of the information intended to identify using a microarray.

Genotyping may be classified into identification and mutation of gene. In a directory at a lower level, is included a disease item relating to a disease, an information of references relating the disease.

Among diseases caused by genetic variation, a simple disease may be caused by one genetic variation. However, in case of a disease (such as cancer) caused by various genetic variations correlated to a plurality of complex genetic information, two or more genes are involved. Therefore, one disease item may have a plurality of gene items at a lower level. Each gene item includes information on references.

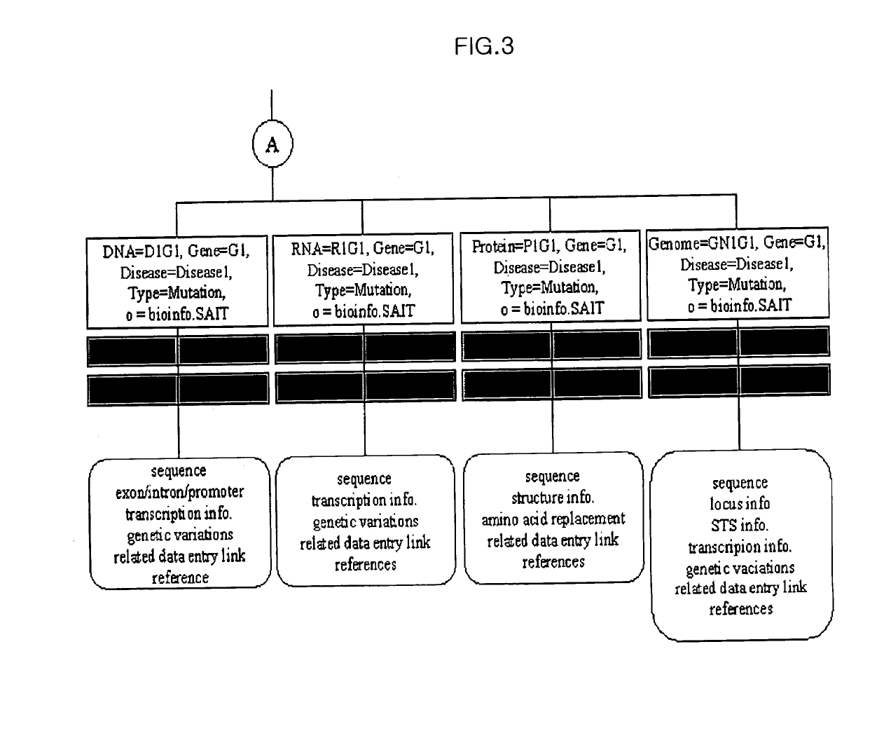

FIG. 3 shows an example of the management of a group of biological information relating to gene items. Each information on DNA, RNA, proteins, and/or genome is included at a level lower than that of the gene item directory.

Genomic item may include information on the other items in a form of annotation.

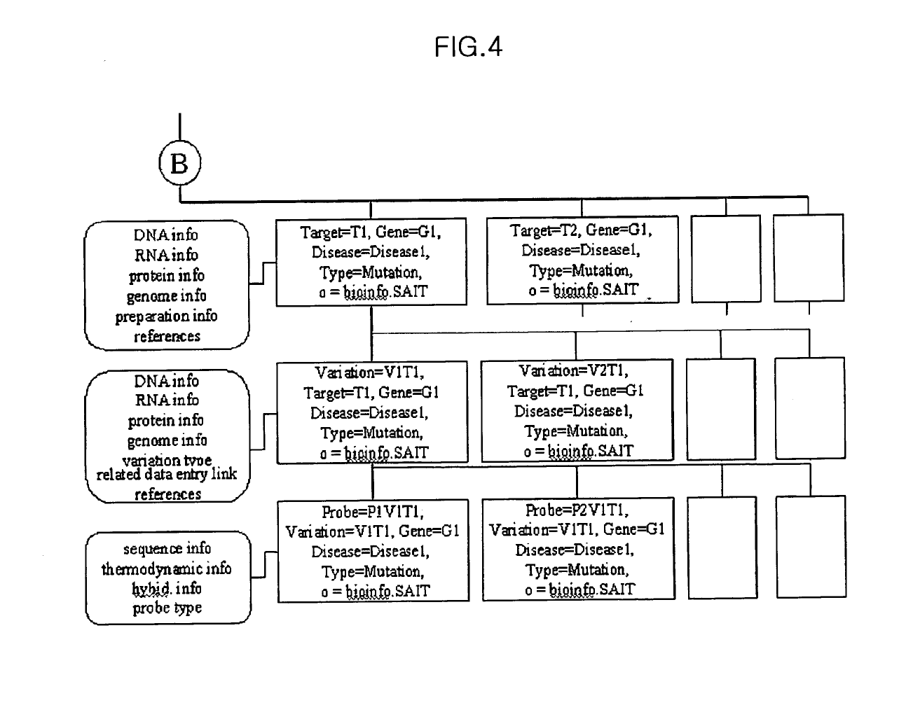

FIG. 4 shows one example for designing a probe for a genotyping-microarray, using the above information. A genetic variation relating to a disease is selected from the above items. A target of a specific region including the genetic variation is determined. The target item includes an experimental method for the preparation of the specific region, along with an information on DNA/RNA/protein/genome items. Each target item includes a variation item showing a genetic variation at a lower level. In the variation, are included all retaining information on variations through the annotation to one target items, while the information on genetic variations in the DNA/RNA/protein/genome items has a meaning of a preliminary investigation. This variation also includes a factor to be considered in designing a probe, such as information on variation type (substitution, insertion, and/or deletion), on variation of a base, and information on whether or not the genetic variation affect a corresponding protein.

A directory of a probe item containing an information on a probe for identifying the specific region is located at a level lower than that of a directory of information on genetic variations. The information on the probe includes a hybridization simulation result (an information on cross hybridization, melting temperature, etc.), a thermodynamic characteristic of the probe (an information such as probe length, hairpin formation energy, self-dimer formation energy, etc.), and/or a base sequence of the probe.

By operating the computer-readable storage medium having the information in directories having a hierarchical structure, the probe information is obtained. And, based on the probe information, a probe having a desired characteristic can be selected. Further, by designing a microarray from the selected probe, a genotyping-microarray is fabricated.

Method and computer system for designing a genotyping-microarray using the computer-readable storage medium according to the present invention have following advantages.

(1) A cost is cut down because a simple structure enables easier management of data.

Because information on a target gene and region of interest thereof are essential factors in genotyping-microarray design, the system of the present invention makes it easier to design and manage a probe used in a microarray. Further, the directory system is more efficient than RDBMS in data reading. Based on the information, the data is searched effectively in an application program for probe design.

(2) Data is completely organized in a hierarchical structure and is easily updated.

In directories, data are managed in a hierarchy-structure, not a relation-based form. Therefore, data used in a genotyping-microarray design is effectively managed, thereby showing an enhanced efficiency in data search, etc. Further, data managed in a hierarchical structure are easily updated.

While this invention has been particularly shown and described with reference to preferred embodiments thereof, it will be understood by those skilled in the art that various changes in form and details may be made therein without departing from the spirit and scope of the invention as defined by the appended claims.

What is claimed is:

1. A computer-readable storage medium having stored thereon:
a first directory comprising an information on DNA, RNA, protein, and/or genome of a target gene;
a second directory comprising an information on a specific region in the target gene; and
a third directory comprising an information on a probe for identifying the specific region,
wherein said first, second, and third directories are organized in a hierarchical structure in which said second directory is at a level lower than that of said first directory and said third directory is at a level lower than that of said second directory.

2. The computer-readable storage medium of claim 1, wherein the target gene includes at least a portion of a gene relating to a disease.

3. The computer-readable storage medium of claim 1, wherein the specific region includes a variation region.

4. The computer-readable storage medium of claim 1, wherein the information on a probe for identifying the specific region comprises information on a base sequence of the probe, a hybridization simulation result, and/or a thermodynamic characteristic of the probe.

5. A method for designing a genotyping-microarray, comprising:
operating computer-readable storage medium having stored thereon: a first directory comprising an information on DNA, RNA, protein, and/or genome of a target gene; a second directory comprising an information on a specific region in the target gene; and a third directory comprising an information on a probe for identifying the specific region, wherein said first, second, and third directories are organized in a hierarchical structure in which said second directory is at a level lower than that of said first directory and said third directory is at a level lower than that of said second directory to obtain an information on a plurality of probes,
selecting a probe having a desired characteristic, based on the obtained probe information, and
forming an microarray comprising the selected probe.

6. The method of claim 5, wherein the information on a probe for identifying the specific region comprises a base sequence of the probe, a hybridization simulation result, and/or a thermodynamic characteristic of the probe.

7. A computer system for designing a genotyping-microarray, comprising:
a processor; and
a computer-readable storage medium of claim 1 accessible by said processor.
